(19) United States
(12) Patent Application Publication
(10) Pub. No.: US 2012/0277807 A1
(43) Pub. Date: Nov. 1, 2012

(54) Methods, Devices and Compositions for Adhering Hydrated Polymer Implants to Bone

(75) Inventors: David Myung, Santa Clara, CA (US); Lampros Kourtus, San Francisco, CA (US); Michael J. Jaasma, San Francisco, CA (US)

(73) Assignee: BIOMIMEDICA, INC., South San Francisco, CA (US)

(21) Appl. No.: 13/542,464

(22) Filed: Jul. 5, 2012

Related U.S. Application Data
(63) Continuation of application No. 12/409,359, filed on Mar. 23, 2009.
(60) Provisional application No. 61/070,305, filed on Mar. 21, 2008.

(51) Int. Cl. A61B 17/84

(52) U.S. Cl. 606/331

(57) ABSTRACT

A method of attaching an implant to a bone, the implant comprising a hydrated polymer comprising a lubricious hydrated surface and an attachment surface comprising accessible chemical functional groups. The method includes the steps of treating the implant or the bone with an isocyanate-containing compound; placing the attachment surface in apposition to the bone; and allowing the isocyanate-containing compound to cure to bond the implant to the bone. The invention also includes a medical implant having a hydrated polymer comprising an attachment surface comprising a thermoplastic material, the hydrated polymer having an interpenetrating polymer network with at least two polymers, the hydrated polymer having a low coefficient of friction on at least one surface.

### CROSS REFERENCE TO RELATED APPLICATIONS

This application is a continuation of pending U.S. application Ser. No. 12/409,359, filed Mar. 23, 2009, which application claims the benefit under 35 U.S.C. §119 of U.S. Provisional Patent Application No. 61/070,305, filed Mar. 21, 2008, the disclosures of which are incorporated by reference as if fully set forth herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

With disease or damage, the normally smooth, lubricious cartilage covering joint surfaces progressively deteriorates, exposing bone and leading to arthritic pain that is exacerbated by activity and relieved by rest. Today, patients with osteoarthritis are faced with only one of two choices: either manage their pain medically, or undergo an effective but highly bone-sacrificing surgery. Medical management includes weight loss, physical therapy, and the use of analgesics and nonsteroidal anti-inflammatories. These can be effective at reducing pain but are not curative. Other options include drugs like glucosamine or hyaluronan to replace the lost components of cartilage, but despite their extensive use in the U.S., their efficacy is still questioned.

When medical intervention fails and a patient's joint pain becomes unbearable, surgery is advised. Total joint arthroplasty is a surgical procedure in which the diseased parts of a joint are removed and replaced with new, artificial parts (collectively called the prosthesis). In this highly effective but invasive procedure, the affected articular cartilage and underlying subchondral bone are removed from the damaged joint. A variety of replacement systems have been developed, typically comprised of ultra-high molecular weight polyethylene (UHMWPE) and/or metals (e.g. titanium or cobalt chrome), or more recently, ceramics. Some are screwed into place; others are either cemented or treated in such a way that promotes bone ingrowth. These materials have been used successfully in total joint replacements, providing marked pain relief and functional improvement in patients with severe hip or knee osteoarthritis.

A large number of patients undergo total hip arthroplasty (THA) in the U.S. each year, which involves implanting an artificial cup in the acetabulum and a ball and stem on the femoral side. The goals of THA are to increase mobility, improve hip joint function, and relieve pain. Typically, a hip prosthesis lasts for at least 10-15 years before needing to be replaced. Yet despite its success as a surgical procedure, THA is still considered a treatment of last resort because it is highly bone-sacrificing, requiring excision of the entire femoral head. It is this major alteration of the femur that often makes revision replacement difficult. While this procedure has a survival rate of 90% or more in the elderly (who usually do not outlive the implant), implant lifetimes are significantly shorter in younger, more active patients. As a result, younger patients face the prospect of multiple, difficult revisions in their lifetime. Revisions are required when implants exhibit excessive wear and periprosthetic bone resorption due to wear particles, as well as aseptic loosening of the prosthesis resulting from stress shielding-induced bone resorption around the implant.

The aforementioned limitations of THA have prompted the industry to seek less bone-sacrificing options for younger patients, with the hope that a THA can be postponed by at least five years or more. One approach towards improving treatment has been to develop less invasive surgical procedures such as arthroscopic joint irrigation, debridement, abrasion, and synovectomy. However, the relative advantage of these surgical techniques in treating osteoarthritis is still controversial. An alternative to THA—hip resurfacing—has now re-emerged because of new bearing surfaces (metal-on-metal, rather than metal-on-polyethylene). While many patients can expect to outlive the procedure's effectiveness, hip resurfacing preserves enough bone stock on the femoral side to allow for later total hip replacement. Unfortunately, there are enough potential drawbacks that doctors offering hip resurfacing say that the procedure should still be deferred as long as possible.

In metal-on-metal resurfacing, the femoral head is shaped appropriately and then covered with a metal cap that is anchored by a long peg through the femoral neck. It requires a more precise fit between the cap and cup, and the procedure generally sacrifices more bone from the acetabulum compared to conventional replacements due to the larger diameter of the femoral component. Furthermore, a resurfacing operation has a steep learning curve and takes longer than a THA. Femoral neck fractures caused by bone resorption around the peg have been reported, and the long-term impact of metal ion release from the bearing surfaces is also not yet known in humans. As a result of these complications, today's resurfacing devices are still only indicated in patients for whom hip pain is unbearable, as is the case for THA.

Joint implants are commonly held in place either by a press-fit mechanism or through a bone cement. In a press-fit mechanism, the implant fits tightly into a space formed by the bone until, over time, new bone is created that holds the implant in place. The implant generally has roughened edges to provide a surface over which cells can migrate and grow. The bone in-growth results in a strong attachment. A press-fit mechanism requires that the bone is strong, healthy and expected to regrow. The procedure is technically demanding, and the fit of the implant has to be precise.

Bone cement is placed between the implant and the bone and can penetrate into the spaces and pores within both the implant and the bone. The bone cement does not adhere, but rather hardens and holds the implant in place. Bone cement may be used with any kind of bone, but is preferred over the press-fit mechanism in cases where the bone is damaged or fragile.

The oldest form of bone cement has been used since the 1960's, and contains polymethymethacrylate (PMMA). PMMA is a colorless liquid that is an irritant to the eyes, skin, and respiratory system. The PMMA is mixed with other components into a paste, and then spread or injected onto the surface of the bone. The temperature rises as it cures which is thought to possibly cause heat related damage to body tissues, including nerves and bone. The PMMA bone cements may cause allergic reactions.

Through time, as metal and plastic implants wear, and the bones and body age and change, implants initially held in place with PMMA bone cement loosen and become unusable or painful. This is especially the case for recipients who are physically active. As a consequence a number of revision surgeries are performed, whereby the implant is removed and a new implant must be put inserted in its place. In addition to the pain and setbacks that arise from undergoing major surgery, the process of removing the old cement can result in additional damage, including bone fractures. The number of knee revision surgeries in the United States is estimated to be at least 22,000 each year. As patients receive their first implant at younger ages, the need for even more revision surgeries can be predicted.

More recently (since about the 1980's), a biodegradable bone cement made with calcium phosphate has been used. Like PMMA, it can be formed into a paste and injected to the bone site. Unlike PMMA, its setting is less predictable, and it takes hours for it to reach maximum strength. Leakage is thought to possibly contribute to tissue damage and nerve pain, and concerns have been expressed over cases of lethal embolization. Implants utilizing the biodegradable bone cement cannot withstand heavy loads, and it is not generally used for knee implants. It is resorbed over time from its outer surface and, ideally, replaced by bone tissue to create a bone-implant linkage.

The attachment of hydrogels to bone has been previously described. For example, U.S. patent application Ser. No. 12/148,534, filed Apr. 17, 2008, describes a hydrogel formed as an interpenetrating network (“IPN”) and its attachment to bone by the application of a precursor solution of reactive monomers or macromonomers that are subsequently polymerized to yield an intervening polymer that is bonded to the bone and is physically entangled and/or chemically bonded with the hydrogel. The disclosure of this application is incorporated herein by reference.

### SUMMARY OF THE INVENTION

The invention relates in general to hydrated polymers, such as hydrogels and hydrogel composites, and orthopedic applications of such hydrated polymers. In particular, the invention relates to methods and compositions whereby hydrated polymers are adhered to mammalian bone or bone-like structures using polyurethane polymers. The invention also relates to the use of thermoformable polymers, such as polyurethane, to attach hydrated polymers to bone or bone-like structures.

One aspect of the invention provides a method of attaching an implant to a bone, the implant comprising a hydrated polymer comprising a lubricious hydrated surface and an attachment surface comprising accessible chemical functional groups. The method includes the following steps: treating the implant or the bone with an isocyanate-containing compound; placing the attachment surface in apposition to the bone; and allowing the isocyanate-containing compound to cure to bond the implant to the bone. In some embodiments, the allowing step includes the step of forming covalent bonds between the implant and the isocyanate-containing compound, such as by delivering UV radiation to the isocyanate-containing compound.

In some embodiments, the allowing step yields a polyurethane or a derivative of a polyurethane. The allowing step may also include the step of creating a non-covalent chemical bond between the implant and the isocyanate-containing compound. In some embodiments, the isocyanate-containing compound may be crosslinked after the allowing step, and in some embodiments the isocyanate-containing compound may be thermoplastic after the allowing step. In some embodiments, the implant includes a crosslinked material.

Some embodiments include the step of applying a solvent (such as, e.g., dimethyl sulfoxide) to the attachment surface to at least partially dissolve the attachment surface and cause a dissolved portion of the attachment surface to flow into the bone.

Some embodiments include the step of swelling the hydrated polymer prior to the treating step. The swelling may take place in an aqueous solution. In some embodiments, the hydrated polymer is partially dried prior to the treating step.

In some embodiments, the isocyanate-containing compound has at least one of a hydroxyl group and an amine group. The isocyanate-containing compound may also have an isocyanate functional group that is part of an aromatic chemical, such as TDI or MDI, and/or an aliphatic chemical, such as IPDI or HDI.

In some embodiments, the treating step includes the step of spreading the isocyanate-containing compound on the attachment surface of the implant and/or immersing at least the attachment surface of the implant in the isocyanate-containing compound. The isocyanate-containing compound may include at least one of an initiator, a catalyst, an accelerator, or an antioxidant.

In some embodiments, at least a portion of bone is removed prior to the placing step. In other embodiments, no bone is removed prior to the placing step. The bone may be part of a joint, such as a hip, shoulder, knee, elbow, finger, toe, wrist, ankle, facet, temporomandibular, intercostal and sternocostal.

In some embodiments, the hydrated polymer includes at least one biomolecule. In such embodiments the biomolecule may be osteoconductive, such as hydroxyapatite, tricalcium phosphate, a bone morphogenetic protein, a growth factor, a glycosaminoglycan, a proteoglycan, collagen, laminin, a bisphosphonate, and any derivatives. The biomolecule may be tethered to the implant.

In some embodiments, the attachment surface has a plurality of spaces so that the isocyanate-containing compound may, e.g., flow into at least one space in the attachment surface prior to the allowing step. In other embodiments, the attachment surface is smooth.

In some embodiments, the treating step includes the step of flowing the isocyanate-containing compound into pores of the bone prior to the allowing step and the allowing step comprises mechanically interlocking the isocyanate-containing compound with the bone. The isocyanate-containing compound may also flow into the implant prior to the allowing step.

In some embodiments, the method includes the step of applying pressure to the implant prior to the allowing step. The implant may include a polyurethane in some embodiments.

In some embodiments, the allowing step includes the step of polymering the isocyanate-containing compound into a biodegradable polymer, resulting in some embodiments in covering 1%-99% of an interface between the attachment surface and the bone with the biodegradable polymer.

Another aspect of the invention provides a method of attaching an implant to a bone, the implant comprising a hydrated polymer comprising a lubricious hydrated surface and an attachment surface comprising a thermoplastic material. The method includes the following steps: placing the attachment surface in apposition to the bone; and applying a stimulus to cause the thermoplastic material to flow into and bond to the bone. In some embodiments, the stimulus may be infrared radiation having a frequency, e.g., close to the resonant frequency of the thermoplastic material. In some embodiments, the stimulus may be a focused light beam, with the thermoplastic material being partially or totally opaque and the hydrated polymer being substantially transparent. The thermoplastic material may also be biodegradable.

Another aspect of the invention provides a medical implant having a hydrated polymer and an attachment surface with a thermoplastic material (such as, e.g., polyurethane), the hydrated polymer including an interpenetrating polymer network with at least two polymers and, optionally, at least one accessible chemical functional group, the hydrated polymer having a low coefficient of friction on at least one surface. The hydrated polymer may also include an ionizable polymer and a neutral polymer, such as a hydrophilic polymer.

In some embodiments, optional the functional groups are selected from the group consisting of carboxylic acid, amine, urethane, and hydroxyl. In some embodiments, the hydrated polymer has at least one of a particle fiber, a particle filler, and a matrix.

In some embodiments, the thermoplastic material may be covalently bonded to a surface of the hydrated polymer. In some embodiments, the thermoplastic material may be a coating on the hydrated polymer. In some embodiments, the thermoplastic material may have hard and soft segments. In some embodiments, the thermoplastic material may be physically entangled with the hydrated polymer. In some embodiments, the thermoplastic material may have a plurality of spaces.

In some embodiments, the hydrated polymer includes at least one of polyurethane, poly(ethylene glycol), poly(acrylic acid), poly(vinyl alcohol), poly(vinyl pyrrolidone), poly(acrylamide), poly(N-isopropylacrylamide), poly(hydroxyethylmethacrylate), a biological polymer, or any derivatives. The hydrated polymer may also have a surface adapted to replace a natural cartilage surface in a mammalian joint, such as a hip, shoulder, knee, elbow, finger, toe, wrist, ankle, facet, temporomandibular, intercostal or sternocostal. In some embodiments, the thermoplastic material may have a surface adapted to conform to such a mammalian joint surface.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-B schematically illustrate attachment of a hydrated polymer implant to a bone according to certain embodiments of the invention.

FIGS. 2A-C schematically illustrate attachment of a hydrated polymer implant to a bone according to certain other embodiments of the invention.

FIGS. 3A-C show attachment of a hydrated polymer implant to a bone and subsequent osteointegration.

FIGS. 4A-C show attachment of a hydrated polymer implant to a femoral head according to certain aspects of the invention.

FIGS. 5A-D schematically illustrate a bonding process according to certain aspects of the invention.

FIG. 6 shows a hydrated polymer implant according to the invention bonded to a model femoral head.

FIG. 7 shows a hydrated polymer implant according to the invention bonded to a model acetabulum.

FIG. 8 shows a hydrated polymer element bonded to a bovine bone sample.

FIGS. 9A-B schematically illustrate thermoplastic bonding of a hydrated polymer implant to bone according to certain aspects of the invention.

FIGS. 10A-B are perspective views showing attachment of hydrated polymer implants to a bone.

FIGS. 10C-E are cross-sectional views of the implant and bone of FIG. 10A showing bone ingrowth over time.

### DETAILED DESCRIPTION OF THE INVENTION

The present invention pertains to a method and composition for adhering hydrated polymers (such as, e.g., hydrogels) to bone and bone-like structures or surfaces. In some embodiments, the hydrated polymer contains accessible chemical functional groups such as amine, hydroxyl, carboxyl, or urethane groups, or combinations of functional groups. It can have a homopolymer, copolymer, semi-interpenetrating or interpenetrating polymer network structure. The invention also pertains to medical implants made with such hydrated polymers and their adhesion to bone and bone-like structures or surfaces. Such medical implants are formed with a lubricious articulating surface designed to replace cartilage and an attachment surface designed for fixation of the implant to bone for use in any joint in the body. The device can be implanted on one side of a joint forming a hydrated polymer-on-cartilage articulation in the mammalian joint. The device could further have a second mating component implanted on the opposing joint surface forming a hydrated polymer-on-hydrated polymer articulation. Alternatively, the device could further have a second mating component implanted on the opposing joint surface forming an articulation between a hydrated polymer on a metal, ceramic, or non-hydrated polymer.

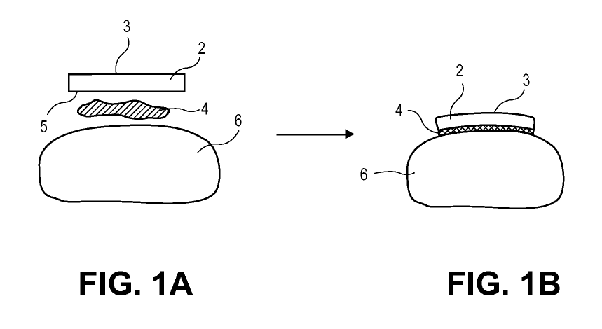

FIGS. 1A-B illustrate one embodiment of the invention. Medical implant 2 having a lubricious, hydrated articulation surface 3 is fixed to bone 6 by means of an isocyanate-containing chemical compound 4 that acts as an intermediary between bone 6 and the attachment surface 5 of the implant 2. In the illustrated embodiment, the isocyanate-containing compound is separate from the implant and can be applied to either the attachment surface of the implant or to the bone. After the implant and bone are brought together and the isocyanate-containing compound is cured and hardened, the implant is fixed to the bone. The mechanism of adhesion of the isocyanate-containing compound 4 and the implant attachment surface 5 is chemical and/or physical, with the chemical adhesion being, e.g., covalent bonds formed between reactive functional groups found on the device material and the chemical groups in the isocyanate-containing compound and/or a variety of non-covalent interactions such as adsorption, hydrophobic interaction, crystallite formation, hydrogen bonds, pi-bond stacking, Van Der Waals interactions, and entanglements between the device and the cured isocyanate-containing compound. In some embodiments, the physical adhesion may be the result of in-filling of spaces, rough areas, surface features and/or pores within the device's attachment surface and within the neighboring bone. In some other embodiments, the attachment surface is smooth. In some embodiments, the isocyanate-containing compound is a pre-prepared mixture of isocyanate chemicals and functionalized molecules with chemical groups that are reactive with the isocyanate such as alcohols, amines, and carboxylic acids, and, in some cases catalysts, accelerators, inhibitors, and/or initiators. The isocyanate-containing compound may be liquid, gel, putty, paste or otherwise flowable.

In some embodiments, polyurethane is used as an intermediary in chemical combination with a hydrated polymer implant for the purpose of promoting both rapid adhesion between hydrated polymer and bone and subsequent osteointegration between hydrated polymer and bone. This dual-action will promote long-term adhesion between a hydrated polymer and bone for the purpose of replacing cartilage. The implant's fixation to bone is based on one or more of the following: (1) Covalent linkages between existing or prepared functional groups on the attachment surface of a hydrated polymer to a polyurethane-based polymer adhesive; (2) interpenetration and physical entanglement between the implant attachment surface and the polyurethane-based polymer network; (3) chemical reaction and/or penetration of bone matrix with the polyurethane-based polymer adhesive; and (4) dual-function of the polyurethane component as both a hydrated polymer implant-to-bone adhesive and a scaffold for additional bony ingrowth and attachment to the hydrated polymer. The polyurethane can be in a variety of forms: resorbable or non-resorbable, porous or non-porous, and used either to completely or just partially cover the interface between implant and bone.

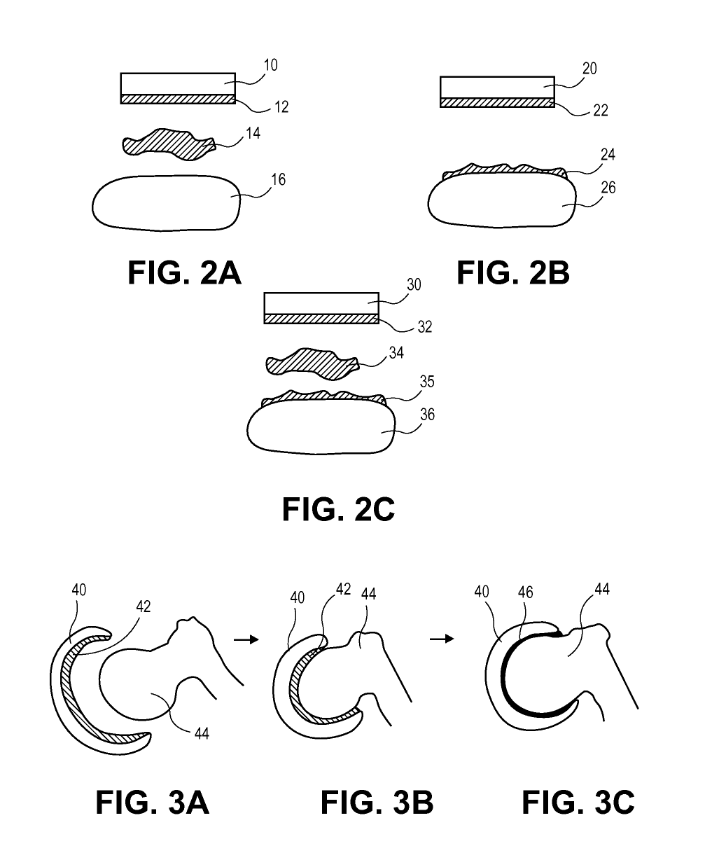

FIG. 2A shows an embodiment in which a hydrated polymer medical implant 10 with a built-in polyurethane-based layer 12 as an attachment surface is attached to a bone or other surface 16 by use of an isocyanate-containing compound 14. After the implant and bone are brought together and the isocyanate-containing compound is cured and hardened, the implant is fixed to the bone.

FIG. 2B shows an embodiment in which a hydrated polymer medical implant 20 with a built-in polyurethane-based layer 22 as an attachment surface is attached to bone by bringing it in apposition to a bone or other surface 26 to which an isocyanate-containing compound 24 has been applied. After curing of the compound 24, the implant 20 and bone 26 are adhered to each other.

FIG. 2C shows an embodiment in which a hydrated polymer 30 with a built-in polyurethane-based polymer 32 as an attachment surface is attached to bone 36 by bringing it and a quantity of an isocyanate-containing compound 34 in apposition to bone 36 to which the same or another isocyanate-containing polymer 35 has been applied. After curing of the compounds 34 and 35, the implant 30 and bone 36 are adhered to each other.

The invention also relates to the use of thermoformable polymers such as polyurethane with useful characteristics such as in situ reactivity and readily tunable mechanical properties, adhesivity, porosity, osteoconductivity, thermoplasticity, and in vivo resorption behavior (i.e. they can be made to be either non-resorbable or resorbable, and at different rates) for the attachment of hydrated polymer implants to bone or bone-like structures. Selective heating of the polyurethane-based polymer adhesive will cause a phase transition from the solid state to a liquid or viscous liquid state so that the polymer layer becomes malleable or flowable. Placing the hydrated polymer implant on the bone, the polymer layer will then enter the interstices and pores of the bone. Removal of the heat or other stimulus will then cause the polymer to harden again, resulting in intimate interdigitation of the polymer with the underlying bone and firm anchorage of the hydrated polymer implant with the bone.

In some embodiments, the polyurethane-based polymer adhesive or thermoformable polymer layer can be biodegradable (resorbable) and used in combination with an implant that supports bone ingrowth. The biodegradable polymer can be applied to the bone or to the implant prior to implantation. After implantation, the degradable polymer is gradually dissolved or resorbed over weeks to years and is replaced by bone, fibrous tissue, or fibrous tissue that becomes bone tissue. The biodegradable polymer can be applied to form a complete intermediate layer between the hydrated polymer implant and the bone so that the hydrated polymer implant and bone do not contact each other directly. Alternatively, the biodegradable polymer can be applied to discrete regions of either the bone surface or the hydrated polymer implant before the implant is implanted on the bone so that the biodegradable polymer covers 1%-99% of the interface between the hydrated polymer implant attachment surface and the bone surface once the implant has been implanted. In this embodiment, the biodegradable polymer provides initial fixation between the hydrated polymer implant and the bone while bone ingrowth occurs into the regions of the hydrated polymer implant that are not covered by the biodegradable polymer. Then, after bone ingrowth has occurred, the biodegradable polymer will gradually degrade. As the polymer degrades, the bone ingrowth now provides the strength of the implant-bone fixation.

The present invention includes arthroplasty implants based on hydrated polymers such as hydrogels that are designed to replace damaged cartilage and to adhere to a particular location within a joint without the need for screws, fixation pins, or other bone-sacrificing means to anchor the device. It further enables rapid adhesion of the hydrated polymer implant to the bone without any substantial preparation or processing of the implant's bone-contacting surface, and then makes bony ingrowth and osteointegration possible to further secure the implant in place, as shown in FIGS. 3. This invention can be used to attach hydrated polymer implants to any joint in the body after removal of diseased or damaged cartilage or cartilaginous structures. These joints include but are not limited to those in the knee, hip, vertebral column (facets or discs in the lumbar or cervical region), elbow, ankle, feet, toes, hands, fingers, wrist, shoulder, temperomandibular joint, sternum, and ribs. In some orthopedic uses, the hydrated polymer implants have a lubricious, relatively low coefficient of friction on its articulating surface, good mechanical properties and is non-resorbable.

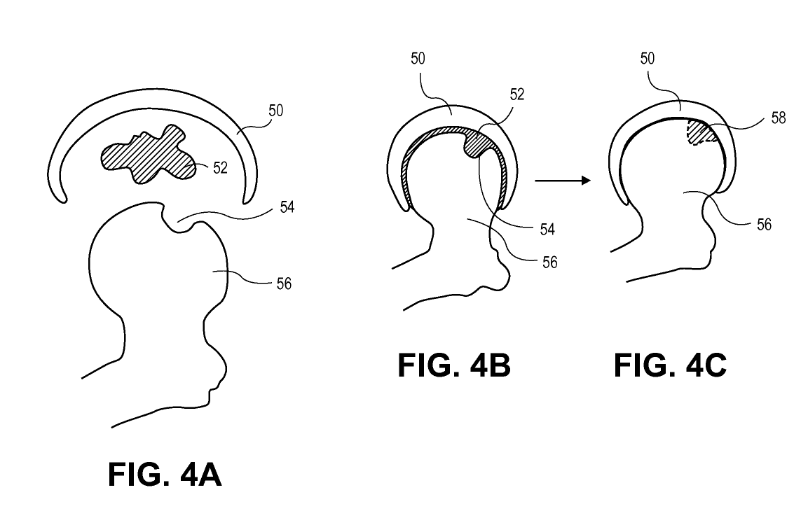

As shown in FIGS. 4A-C, the materials described herein can be used to resurface necrotic joints, joints containing cysts, and/or joints with bone that has collapsed. They can also be used following bone-sacrificing arthroplasty. In one embodiment, an implant 50 comprised of a hydrated polymer with a thin layer of polyurethane based adhesive is placed over the normal joint region. An additional volume of polyurethane-based adhesive 52 fills in the voids in the underlying bone. The polyurethane-based adhesive acts as a bone scaffold. Over time, bone will grow into the cured polyurethane-based polymer (as shown in region 58 in FIG. 4C), which helps restore the bone morphology and serves to anchor or secure the hydrated polymer implant to the bone.

One embodiment of the invention provides: (1) simultaneous reaction at the hydrated polymer implant's attachment surface and polymerization/crosslinking of the isocyanate-containing compound in the adjacent space during curing, (2) interpenetration of the resulting polyurethane polymer within the hydrated polymer network, and (3) in-filling of the adjacent bone with the polyurethane.

Another embodiment of the invention provides: (1) coating of the implant's attachment surface with a polyurethane coating during manufacturing or prior to implantation to create a hydrated polymer implant with a polyurethane-based coating or layer on the intended bone-contacting attachment surface, (2) subsequent preparation of the bone with the same or different polyurethane-based adhesive polymer, (3) apposition of the polyurethane-modified hydrated polymer implant with the polyurethane-coated bone, (4) adhesion between the polyurethane-coated aspects of the hydrated polymer implant and bone by chemical reaction and/or mutual in-filling. The resulting continuous polyurethane-based polymer(s) adhere the hydrated polymer implant to bone by filling in interstices and pores within subchondral or trabecular bone and also of the implant (if the surface of the implant is also porous).

The hydrated polymer implant can have, at least in part, a homopolymer, copolymer, semi-interpenetrating or interpenetrating polymer network hydrogel structure. Examples of polymers for the hydrated polymer implant include but are not limited to poly(ethylene glycol), poly(acrylic acid), poly(vinyl alcohol), poly(vinyl pyrrolidone), poly(acrylamide), poly(N-isopropylacrylamide), poly(hydroxyethylmethacrylate), polyurethanes, biological polymers (e.g. collagen, hyaluran or chitosan) and derivatives and combinations thereof The hydrogel can be an interpenetrating or semi-interpenetrating polymer network having two polymers such as poly(ethylene glycol)-diacrylate/poly(acrylic acid) (PEG/PAA) or poly(vinyl alcohol)/poly(acrylic acid) (PVA/PAA) or alternatively polyurethane/poly(acrylic acid). The hydrogel can be an interpenetrating network having a first and second polymer. The first polymer can be a thermoplastic with high mechanical strength, including but not limited to polyurethane (PU) (including but not limited to Elasthane® 55D or other polyether urethanes, polycarbonate urethane, polycarbonate urethane urea, silicone polyether urethane, polyurethane urea, and silicone polycarbonate urethanes); acrylonitrile butadiene styrene (ABS); polylactic acid (PLA); polysulfone (PSU); polyvinyl acetate (PVA). The second polymer can be a hydrophilic polymer derived from ionizable, vinyl monomers, including but not limited to a carboxylic acid containing vinyl monomer, such as acrylic acid and methacrylic acid, or a sulfonic acid-containing vinyl monomer, including but not limited to 2-acrylamido-2-methylpropanesulfonic acid, sulfopropyl acrylic acid ester, hyaluronic acid, heparan sulfate, and chondroitin sulfate. The second monomer could also be non-ionic, such as acrylamides, N-isopropyl acrylamide, methyl-methacrylate, N-vinyl pyrrolidone, 2-hydroxyethyl methacrylate, 2-hydroxyethyl acrylate or combinations or derivatives thereof Copolymers of ionic and non-ionic monomers can also be used. Furthermore, crosslinked linear polymer chains as well as biomolecules such as proteins and polypeptides (collagen, hyaluronic acid, or chitosan) can be used. For methods of making suitable hydrated polymers and hydrogels, see copending U.S. patent application Ser. No. 12/148,534 (filed Apr. 17, 2008); No. 61/079,060 (filed Jul. 8, 2008); No. 61/086,442 (filed Aug. 5, 2008); and No. 61/095,273 (filed Sep. 8, 2008), the disclosures of which are incorporated herein by reference.

In some embodiments, the hydrated polymer contains accessible and reactive functional groups such as carboxyl, urethane, amine, or hydroxyl groups, or combinations of functional groups. These functional groups can react selectively with isocyanates contained in the adhesive to form urethane or urea bonds. One particular kind of hydrated polymer useful for this invention is an interpenetrating polymer network composed of poly(acrylic acid) and another hydrophilic polymer network. Poly(acrylic acid) contains an abundance of carboxylic acid side groups. One example of such a hydrated polymer is a hydrogel comprised of a poly(ethylene glycol)-diacrylate/poly(acrylic acid) (PEG/PAA) interpenetrating polymer network made by two-step sequential photopolymerization with or without crosslinking of PAA by a bifunctional or multifunctional crosslinking agent (e.g., triethylene glycol dimethacrylate or methylene bisacrylamide) by methods described in the art. Gamma irradiation or electron beam radiation can also be used to crosslink the hydrated polymer implants. Other hydrogels with different functional groups (such amine or hydroxyl groups) or combinations of functional groups may also be used.

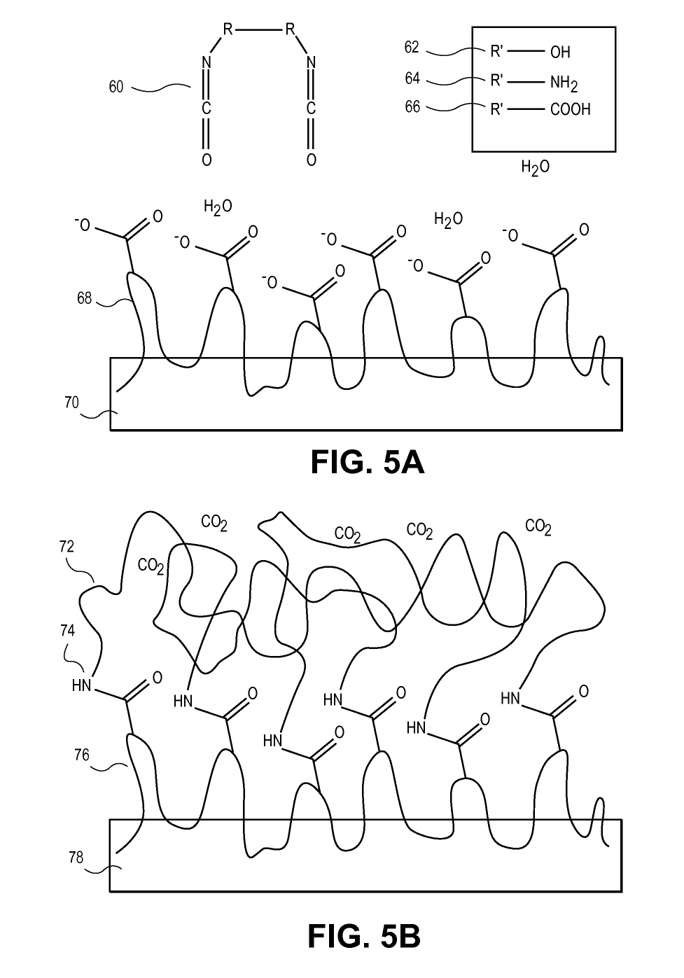

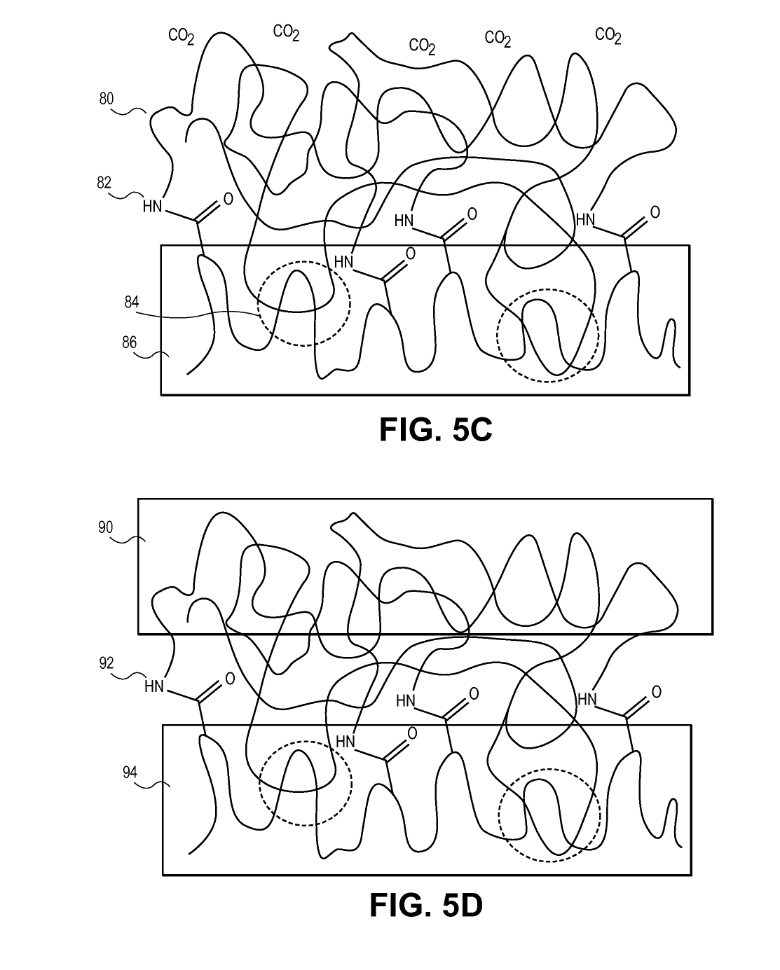

As shown in FIGS. 5, isocyanate groups in the isocyanate-containing compound can react with a number of different functional groups on a hydrated polymer surface. For instance, if poly(acrylamide) and PAA are used, then the amine groups in the acrylamide and the carboxylic acid groups in the PAA can both react with the isocyanates to form links with an adjacent polyurethane. If poly(vinyl alcohol) is used, then a hydroxyl will be reacted with isocyanate instead of amines along with the carboxylic acids. Another combination is that between a poly(vinyl alcohol) and a poly(acrylamide), in which case hydroxyls and amines are reacted with isocyanates. Isocyanate can also react with amine groups on an implant surface to yield a substituted urea linkage. Isocyanates can also react with urethane groups on an implant surface to yield allophanate linkages. These coupling reactions can work in concert with each other and by inter-diffusion and entanglement of the polyurethane-based polymer resulting from the curing of the isocyanate-containing compound and the hydrated polymer implant. These additional physical entanglements may reinforce the adhesion between the two materials. It may also act alone, without any covalent linkages, and be reinforced solely by non-covalent interactions such as hydrogen bonding, hydrophobic interactions, crystallization, pi-bond stacking, or Van Der Waals interactions.

In some embodiments, the bonding process involves a chemical reaction between functional groups accessible on a hydrogel surface and an isocyanate-containing compound as illustrated in FIGS. 5A-D. As shown in FIG. 5A, a diisocyanate chemical 60 is reacted with another polymer or polymers (e.g. a soft segment and a chain extender) containing either hydroxyl functional groups 62, amine functional groups 64, and/or carboxylic acid functional groups 66. When reacted together in the presence of carboxylic acid functional groups 68 present on an existing hydrated polymer 70, a chemical reaction occurs that results in the composition shown in FIG. 5B.

In FIG. 5B, several structures result: a polyurethane-based polymer 72 (with carbon dioxide byproducts), amide linkages 74 between polymer 72 and the polymer backbone chains 76 present at the hydrogel surface 78. Another configuration is shown in FIG. 5C in which the polyurethane-based polymer 80 is attached through both amide linkages 82 and physical entanglements 84 to the adjacent hydrated polymer 86. FIG. 5D shows how an object such as bone 90 is attached though a polyurethane-based polymer 92 to an adjacent hydrogel 94 by the processes described herein.

In one example, according to an embodiment of the invention, an isocyanate-containing compound comprising poly(tetramethylene oxide) diol and methylene diphenyl diisocyanate was reacted with butanediol in a prepolymer solution and carboxylic acid groups on a hydrogel surface. The result was a polyurethane-based polymer bonded to the hydrogel, with the polyurethane polymer and hydrogel being physically entangled and/or covalently linked through amide linkages resulting from the reaction between isocyanate groups in the polyurethane precursor and the carboxylic acid groups on the hydrogel. When cast in direct apposition with bone, the polyurethane polymer also adhered to bone and served as an intervening adhesive layer between the hydrogel and bone.

In another example, to prepare a PEG/PAA hydrogel (swollen in phosphate buffered saline), the surface was dabbed dry and then gently air-dried using a heat gun or compressed air. This hydrogel had an abundance of poly(acrylic acid) polymer chains present at its surface. The isocyanate-containing compound (containing the methylene diphenyl diisocyanate and diol prepolymer mixture) was then spread over the surface of the hydrogel, then the hydrogel was placed adhesive-side down on bone, and allowed to cure for several minutes while pressure was applied to keep the hydrogel firmly pressed against the bone. Curing of the adhesive continued to completion in several hours. The result was a polyurethane urea layer formed and chemically adhered to the surface of the hydrogel and physically interlocked with bone.

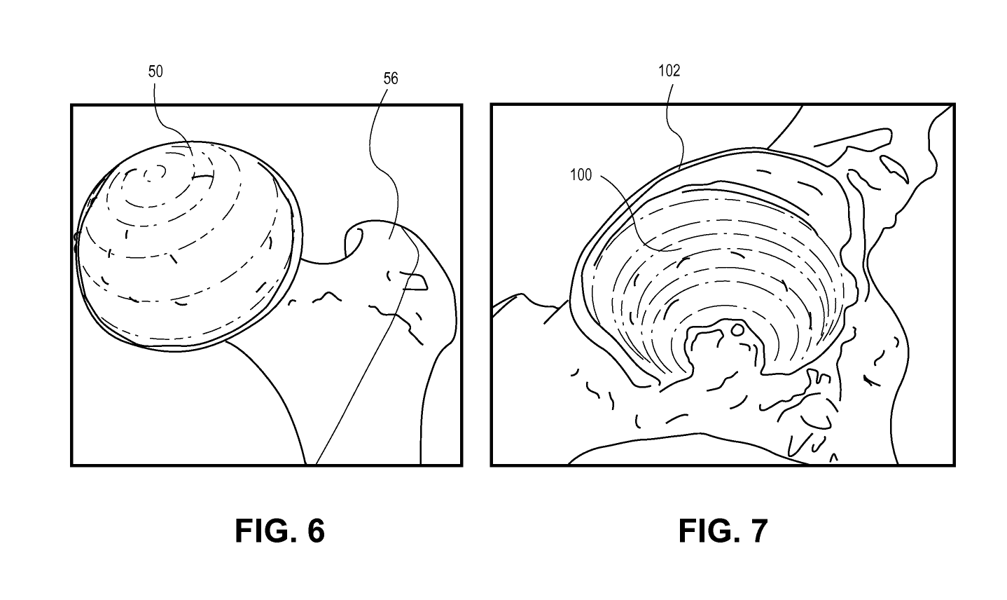

The methods, devices and compositions of this invention can be used to bond hydrated polymers and hydrogels to a variety of surfaces. PEG/PAA hydrogels were bonded to various objects using the present invention. In one example, shown in FIG. 6, a hemisphere-shaped hydrogel 50 was bonded to a femoral head of a model femur 56 using a polyurethane-based polymer (not shown) beneath the hydrogel cap 50. The implant process is shown schematically in FIGS. 4A-C. The hydrogel 50 and a polyurethane-based adhesive polymer 52 are placed over the surface of the femoral head. The polymer 52 may fill in any defect 54 in the femoral head in addition to adhering the hydrogel 50 to the femoral head. Over time, the bone grows into the filled-in defect by osteointegration, as shown schematically in FIG. 4C.

In another example, shown in FIG. 7, a semilunar-shaped hydrogel sheet 100 was bonded to a model acetabulum 102. Details of various hydrogel-based implants may be found in U.S. patent application Ser. No. 12/148,534.

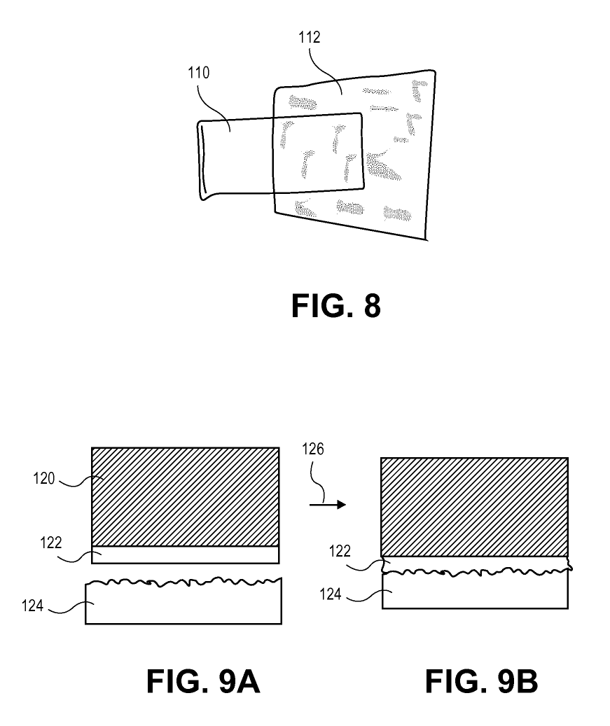

In yet another example, shown in FIG. 8, a PEG/PAA hydrogel 110 was bonded to bovine bone samples 112. These samples were subjected to lapshear and peel tests using a uniaxial materials testing apparatus. The lapshear test showed a bond shear strength of 60 kPa, and the peel test showed a peel strength of up to 0.08 N/mm. Lapshear tests were also conducted on the bond strength between polycarbonate urethane and bovine cancellous bone when using a polyurethane adhesive. The shear strength ranged from 0.21-0.68 MPa. These lapshear and peel experiments on these hydrated polymer-bone specimens demonstrate the loads that were necessary to separate the hydrated polymers from the bone due to the strength of polyurethane-based adhesive. This demonstrates adhesion between the hydrated polymer and the polyurethane adhesive as well as between the polyurethane adhesive and bone.

Bonding of the non-lubricious polycarbonate urethane to bone via polyurethane-urea adhesive demonstrates the ability to bond the attachment surface of the implant to bone. In order to bond a complete device, comprised of one lubricious, hydrated surface and one attachment surface, to bone, the implant device is first fabricated. In one embodiment, the implant is composed of polyurethane. One surface of the implant is modified with poly(acrylic acid) to create an interpenetrating polymer network of polyurethane and poly(acrylic acid). This surface is swollen with water to create a lubricious, hydrated surface. A second surface of the implant, the attachment surface, remains dehydrated. The polyurethane-based adhesive is applied to the attachment surface, and the device's attachment surface is placed against a bone surface. The adhesive is allowed to cure, resulting in a bond between the bone and the attachment surface of the implant.

In one embodiment, the addition of the polyurethane material takes place after the hydrogel is manufactured. In another embodiment, the polyurethane is added to the hydrogel at the same time the hydrogel is being manufactured. The polyurethane may be preformed, such as a commercially available polycarbonate polyurethane such as Bionate® 75D, or it may be synthesized during manufacture (or implantation).

The intermediary layer may be formed from a pre-polymer precursor solution containing one or more chemicals each having at least two isocyanate groups, and usually two other chemicals (a soft segment and a chain extender) each typically containing at least two hydroxyl groups (diol compounds) that form the basis of a polyurethane structure. The polyurethane can have any type of hard segment, soft segment, or chain extender. Examples of soft segments include but are not limited to polyethylene oxides, polypropylene oxides, polytetramethylene oxides (PTMO), poly(dimethylsiloxane), and polycarbonate, and derivatives and combinations thereof. The isocyanate can be either aliphatic (e.g. hexamethyl diisocyanate (HDI) or IPDI) or aromatic (e.g. MDI or TDI). An initiator, catalyst, or accelerator can be added to speed up the curing reaction. A list of different hard and soft segments and chain extenders may be found in Polyurethanes in Biomedical Applications (Nina M. K. Lamba, Kimberly A. Woodhouse, Stuart L. Cooper, Michael D. Lelah, CRC press 1987), the disclosure of which is incorporated herein by reference.

Antioxidants may be added to improve the long-term in vivo stability of the polyurethane-based polymer. In one embodiment, the polyurethane intermediary is configured to act as a scaffold for cell in-growth and bone matrix deposition.

The polyurethane based polymer can be impregnated or tethered with biomolecules, including but not limited to osteoconductive biomolecules such as hydroxyapatite, carbonated apatite, tricalcium phosphate, bone morphogenetic proteins (BMPs), growth factors, glycosaminoglycans, proteoglycans, collagen, laminin, and bisphosphonates, as well as derivatives and combinations thereof.

In some embodiments, the bone-interfacing region (bearing layer) is capable of binding to calcium-containing and phosphate-containing bone-matrix constituents of the bone. The bearing layer (bone interfacing layer) can be anchored to a synthetic bone-like structure, such as a porous calcium-phosphate containing material, including but not limited to porous carbonated apatite, beta-tricalcium phosphate, or hydroxyapatite).

In some embodiments, as mentioned above, porosity on the device is desired to facilitate bone ingrowth. One way to achieve this is to alter the hydrophilic/hydrophobic ratio of the polyurethane to produce variations in the adhesive and bony ingrowth capacity of the material. The polyurethane-based polymer can be modified to be more or less hydrophilic or hydrophobic, and with a greater or lesser capacity to expand or swell. It can be modified to crystallize or foam and create pores (open or closed cell) or have no pores at all. The hydrogel can be reinforced with particle fibers or particle fillers. Porosity can be created either through the use of foaming agents, through controlled reaction with water (to yield carbon dioxide), or through the use of porogens which are encapsulated and then washed away (such as salt or sugar crystals or polymer particles). The hydrated polymer implant may be reinforced with particle fibers or particle fillers.

In another example, the implant attachment surface is characterized by having a porosity or surface roughness on the order of 10 to 2000 microns, porosity of 15-70%, and a compressive strength exceeding 1 MPa to accommodate tissue ingrowth/integration and bone formation. The bone interfacing region can be comprised of sinterered polycarbonate urethane beads. Briefly, particles (size range 250-1500 um) of polycarbonate urethane, including but not limited to Bionate® 55D, Bionate® 65D, and Bionate® 75D, are sintered in a mold using heat (220-250° C.), pressure (0.001-100 MPa) and/or solvent for 10-30 minutes. The beads may be made by any process, such as those described in Brown et al., Journal of Biomedical Materials Research Part B: Applied Biomaterials, “Solvent/Non-solvent sintering: A novel route to create microsphere scaffolds for tissue regeneration,” 2008; 86B(2):396-406 or Borden et al., Journal of Biomedical Materials Research 2002; 61 (3):421-9, “The sinterered matrix for bone tissue engineering: in vitro osteoconductivity studies.” The bone-interfacing region could also be pre-coated with calcium-containing and phosphate-containing constituents. In still another example, biomolecules could be chemically or physically bonded to the bone-interfacing region. The porous construct is used with an overlying bearing surface made from any of the lubricious polymers.

Any formed polyurethane intermediary that is attached to or co-mingled with the hydrated polymer implant can be attached to the surface of the bone, bone-like surface, or cartilage. In one embodiment, the formed intermediary can be caused to selectively soften, melt or, flow into the pores or interstices of subchondral or trabecular bone with mechanical vibrations (vibrational welding), ultrasonic energy (ultrasonic welding), high frequency electromagnetic energy (radiofrequency (RF) welding; and microwave welding), laser beam energy, infrared (IR) energy, selective spectrum infrared (IR) energy, light (UV or visible) and heat. After the energy source is removed, the material resolidifies. This process is shown schematically in FIG. 9, in which a hydrated polymer implant 120 with a thermoflowable layer 122 is adhered to bone 124 via the application of energy 126. The thermoflowable material also may have embedded additives such hydroxyapatite, radio-contrast agents, or metallic particles that allow for faster heating of the material.

In one embodiment, the polyurethane intermediary layer is selectively softened or melted via selective infrared excitation. Segmented polyurethanes contain various hard and soft segments each of which may hold a characteristic chemical bond that can be selectively resonated with infrared radiation of appropriate frequency. As such, a polycarbonate urethane (e.g. Bionate® 75D) can be used as an intermediary layer between the hydrated polymer implant and the bone. This polyurethane holds a carbon-oxygen double bond which presents peak absorption in infrared spectroscopy at 1550-1750 cm⁻¹. Delivering infrared radiation at that frequency will cause the material to heat and therefore liquify therefore achieving penetration in the porosity of the bone and mechanical interlocking after the material cools and solidifies. The selective excitation is required in order to avoid heating the rest of the hydrated polymer implant (and thus cause it to soften or melt); therefore the excitation frequency should not be close to the excitation frequencies of the molecular bonds of the hydrated polymer implant. Cooling of the device will then cause the intermediary material to harden again, thereby anchoring the device.

In another embodiment, the intermediary layer has a melting or softening temperature that is lower than that of the hydrated polymer implant that comprise the device. In this case, heating the device above the melting/softening temperature of the intermediary layer but below the melting temperature of the hydrated polymer implant will cause selective melting therefore achieving penetration in the porosity of the bone and mechanical interlocking after the material returns to a below melting/softening temperature. Heating of the device can be achieved with a heating element in the proximity of the device just before implantation. Combinations of heating and mechanical pressure can also be applied in order to induce permanent creep of the intermediary material according to the impression of the bone microstructure, and therefore increase the mechanical interlocking.

In another embodiment, the intermediary layer has such optical (i.e., opacity) and thermal properties that a focused laser beam can selectively soften or melt it without melting the rest of the hydrated polymer implant. In such an embodiment, the substantially transparent hydrated polymer implant is placed on the bone, and a handheld apparatus that delivers focused laser energy pulses locally heats the partially or totally opaque intermediary layer to cause selective softening or melting therefore achieving penetration in the porosity of the bone and mechanical interlocking after the material returns to below melting or softening temperature. The laser light frequency may be also dialed in so that it is close to the resonant frequency of one or more atomic or molecular bonds of the intermediary layer material.

In one embodiment, the intermediary layer can have such geometric features that facilitate the thermal transition (or phase transition) by requiring lower energy levels to be delivered. These geometric features can be pillars, bumps/pores, grooves or other extrusions/protrusions that increase the surface area but also reduce the volume of the affected (softened or melted) region.

In another embodiment, the intermediary layer can be temporarily dissolved using an appropriate solvent causing it to soften or even flow. For polyurethanes, such a solvent may be Dimethyl Sulfoxide (DMSO). The application of the solvent can be done several minutes before implantation to allow for partial dissolving of the intermediary layer and thus cause it to penetrate in the porosity of the bone and achieve mechanical interlocking after removal of the solvent and subsequent solidification of the polyurethane.

The hydrated polymer implant can be molded to the shape of existing joint structures or be inserted into prepared crevices in the bone in the shape of plugs, discs, sheets, caps, cups, as well as non-symmetric shapes such as those found in mammalian joints. In the case of plug or sheet, it can be used to partially repair focal or partial defects in joint cartilage (instead of resurfacing the entire joint). The polyurethane-based adhesive would anchor the hydrated polymer implant to the bone in a configuration that allows the hydrated polymer implant to act as a bearing or protective surface.

The polyurethane-based polymer can be fully degradable, partially degradable, or non-degradable. It can also be laced with a non-degradable, flexible matrix of any material to aid the anchoring and/or osteointegration process. It can also be built (polymerized) at the same time and continuously with the said hydrated polymer implant, to form a composite structure with hydrated polymer implant on at least one side and polyurethane-based polymer on at least one other side.

The intermediary fixation material, i.e. polyurethane-based adhesive or thermoflowable material, can, after any hardening and curing, be biodegradable with biocompatible degradation products so that it is gradually replaced by bone, fibrous tissue, or fibrous tissue that is converted to mineralized tissue. By selecting the polymer composition (i.e. co-polymers composition, polymer blend ratio, crystallinity via hard:soft segment ratio) and biostability (i.e. hydrolytic and oxidative stability) characteristics, the mechanical and biodegradative properties of the polymer can be tuned to initially provide a high-strength bond between the hydrated polymer implant and the bone while allowing for degradation of the polymer, and the related decline of mechanical properties, at a desired time (weeks to years) after implantation. The desired time to degradation can vary from weeks in minimally load-bearing applications (some peripheral joints) to months or years in high-impact joints (e.g. knee and ankle) or in osteoporotic patients and patients with pathologies that decrease bone formation capabilities. Examples of degradable polyurethane compositions useful as the polyurethane intermediary are incorporated herein through the following citations: Gorna K, Gogolewski S. Preparation, degradation, and calcification of biodegradable polyurethane foams for bone graft substitutes. J Biomed Mater Res A. 2003 Dec. 1; 67(3):813-27.; Scott A. Guelcher, Vishal Patel, Katie M. Gallagher, Susan Connolly, Jonathan E. Didier, John S. Doctor, Jeffrey O. Hollinger. Tissue Engineering. May 2006, 12(5): 1247-1259. doi:10.1089/ten.2006.12.1247., Synthesis of biocompatible segmented polyurethanes from aliphatic diisocyanates and diurea diol chain extenders, Acta Biomaterialia, Volume 1, Issue 4, July 2005, Pages 471-484).

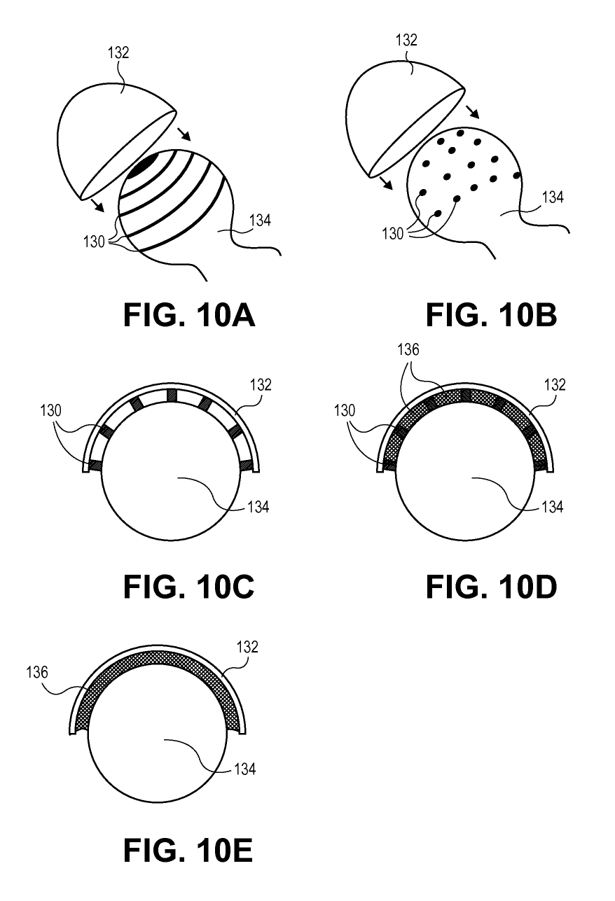

In another embodiment, illustrated in FIGS. 10A-E, a biodegradable intermediary fixation material can be used in a hybrid fixation technique where the fixation material and bone ingrowth are combined to form a temporally constant, high-strength adhesion between the hydrated polymer-based implant and the bone. In this embodiment, the intermediary fixation material 130 is applied to portions of the interface, but not the entire interface, between the hydrated polymer-based implant 132 and the bone 134 in, e.g., discrete points, lines, and geometric patterns that are regularly or randomly oriented across the implant-bone interface. For example, FIG. 10A shows the application of the intermediary fixation material 130 in rings, and FIG. 10B shows the application of the intermediary fixation material 130 in an arrangement of points or dots. Thus, the intermediary fixation material covers a total of 1%-99% of the interface, and the remaining regions of the interface, where the fixation material is absent, have direct contact between the hydrogel-based implant and the bone. During implantation, the intermediary fixation material can be applied to the bone, the implant, or both before the implant is implanted.

In embodiments, the intermediary fixation material is a biodegradable polymer that provides initial fixation between the hydrated polymer implant and the bone while bone ingrowth occurs into the regions of the hydrated polymer implant that are not covered by the biodegradable polymer. Then, after bone ingrowth 136 has occurred, the biodegradable polymer gradually degrades, as shown in FIGS. 10D-E. As the polymer degrades, its mechanical properties decline, and the new bone tissue that has been formed via bone 136 ingrowth into the implant now provides the strength for implant-bone fixation.

What is claimed is:

1. A method of attaching an implant to a bone, the implant comprising a semi-interpenetrating network hydrogel structure comprising polyurethane and poly(acrylic acid), the implant comprising a lubricious hydrated surface comprising hydrated poly(acrylic acid) within the semi-interpenetrating network hydrogel structure and an attachment surface comprising polyurethane, the method comprising:
applying a polyurethane-based adhesive to the implant attachment surface or to the bone;
placing the attachment surface in apposition to the bone; and
after the placing step, allowing the polyurethane-based adhesive to cure to bond to the implant and to the bone.

2. The method of claim 1 wherein the allowing step comprises delivering light energy to the adhesive.

3. The method of claim 2 wherein the light energy comprises UV light.

4. The method of claim 1 wherein at least a portion of bone is removed prior to the placing step.

5. The method of claim 1 wherein no bone is removed prior to the placing step.

6. The method of claim 1 wherein the bone is part of a joint.

7. The method of claim 6 wherein the joint is selected from the group consisting of hip, shoulder, knee, elbow, finger, toe, wrist, ankle, facet, temporomandibular, intercostal and sternocostal.

8. The method of claim 1 wherein the semi-interpenetrating network hydrogel structure further comprises at least one biomolecule.

9. The method of claim 8 wherein the at least one biomolecule is osteoconductive.

10. The method of claim 8 wherein the at least one biomolecule is tethered to the implant.
